(19) United States
(12) Patent Application Publication
(10) Pub. No.: US 2006/0175219 A1
(43) Pub. Date: Aug. 10, 2006

(54) Package for articles having dual opening system

(76) Inventors: Emma Somma, Frankfurt (DE); Christian Kohlweyer, Bad Vilbel (DE); Ibrahim Ulas, Oberursel (DE)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/330,531

(22) Filed: Jan. 12, 2006

(30) Foreign Application Priority Data
Feb. 4, 2005 (EP) 05002438.9
Dec. 21, 2005 (EP) 05112669.6

(51) Int. Cl. A61B 19/02

(52) U.S. Cl. 206/440

(57) ABSTRACT

A flexible package for a multiplicity of articles. The package includes a refastenable flap as first opening member. The package also includes a secondary opening member. The package provides improved hygienic protection of the articles remaining in the package after its opening.

### FIELD OF THE INVENTION

The present invention relates to a package including a refastenable flap as first opening means and underneath the flap a secondary opening means. The package provides improved hygienic protection of the articles remaining in the package after its opening.

### BACKGROUND OF THE INVENTION

Many packages are known in the art. Due to the high hygienic requirements of sanitary absorbent articles, like sanitary napkins, panty liners, incontinence pads or diapers, some of such articles are packaged individually within an outer package. This requires a high amount of packaging material. Therefore attempts have been made to develop packages for such sanitary articles in order to save material. Examples of packages for stacks of absorbent articles are described in WO-A-02/39943 or WO-A-02/085271, both having a reclosable flap. However, packages of this kind are not completely sealed against dust, moisture and other contamination. Therefore, especially while those packages are in the warehouse or on the shelves of a retailer, the content of such a package is to a certain extent exposed to contamination. The contamination of the stored absorbent articles can be prevented by individually wrapping them. However, this undesirably consumes extra material.

Therefore, a need exists to provide a package for a multitude of absorbent articles, which is reclosable and which protects the articles contained therein from contamination during storage and shelf life without the need for further packaging or wrapping material. It would be desirable for such a package to have convenient opening. It would also be desirable for such packages to provide for easy dispensing of the products contained therein.

### SUMMARY OF THE INVENTION

The present invention addresses the above problem by providing a package for a multiplicity of absorbent articles arranged in at least one stack, the package comprising a main package body and a reclosable flap. The main package body encloses the at least one stack. The reclosable flap is hingedly connected to the main package body along a line of connection, wherein the flap can be pivoted between an open and a closed configuration. The package further comprises a secondary opening means being capable of providing an opening in the main package body, wherein the secondary opening means is arranged in relation to the line of connection such that the flap in the closed configuration covers the opening provided by the secondary opening means.

### BRIEF DESCRIPTION OF THE DRAWINGS

FIG. 1 illustrates a package of the present invention with the flap is in its closed configuration.

FIG. 2 illustrates a package of the present invention with the flap in its open configuration. The secondary opening member is exposed.

FIG. 3 illustrates a package of the present invention with the flap in its open configuration and the secondary opening member partially opened, thereby exposing the essentially linear opening. It is visible that two stacks of absorbent articles are contained in the main package body.

### DETAILED DESCRIPTION OF THE INVENTION

The term “absorbent article” is used herein in a very broad sense, including, but not limited to, any article suitable for receiving and/or absorbing and/or containing and/or retaining body fluids. The absorbent article of the present invention typically comprises a structure having a fluid pervious topsheet as the wearer-facing layer, a fluid impervious backsheet that may be water vapour and/or gas pervious as the garment-facing layer and an absorbent core element comprised there between. Certain absorbent articles in the context of the present invention are disposable absorbent articles, for example, articles for feminine protection like incontinence pads, sanitary napkins, tampons, breast pads or panty liners. Other suitable absorbent articles are incontinence pads, diapers, and perspiration pads. The absorbent articles can be individually packaged or can alternatively be without individual packaging. The absorbent articles in the package can be arranged in one or more stacks in any suitable manner.

The term “disposable” is used herein to describe absorbent articles, which are not intended to be laundered or otherwise restored or reused as an article (i.e. they are intended to be discarded after a single use and preferably to be recycled, composted or otherwise disposed of in an environmentally compatible manner).

The term “stack” herein refers to a multitude of articles being arranged behind one another or on top of each other in an essentially coextensive manner. The package of the present invention contains at least one, and optionally multiple, stacks of absorbent articles.

The term “dust proof” herein means that the package is sealed such that solid contaminants cannot access its interior while the package is sealed. In certain embodiments, the package is free of perforations and the like, because these could allow solid contaminants access the package. Also, tamper proofness is a consumer benefit especially for hygiene products. A sufficient degree of dust proofness can also be achieved with the secondary opening member comprising perforations, because these perforations will be covered by the primary opening member, i.e. the flap, and thus dust and other contaminants will be significantly hindered from accessing the interior of the package.

The main package body is made from suitable material, which offers the required degree of hygienic protection to the articles stored therein. The package of the present invention may be made of flexible polymeric film material. Exemplary suitable materials are PE (polyethylene) and derivatives like ethylen-vinylacetate-coploymers, polypropylene (PP), polyester, polystyrol (PS), cellophane, polyvinylchloride (PVC), polyvinylalcohol (EVOH), polyvinylacetal (e.g. PVB), flouropolymers, polyethylenterephtalat (PET), polycarbonat (PC), polyamide (PA), polyphenylenoxid, polysulfon, Polyurethanes and the like. Also encompassed are biodegradable polymers based on aliphatic or aromatic polyesters, starch or similar materials that are suited for flexible film production. This also includes laminated or co-extruded material made from above materials. Furthermore, suitable materials for making the main package body are materials comprising nonwovens and/or paper, provided they are or can be treated to be substantially impervious to contaminants such as dust. Examples are laminates of nonwovens and polymeric films or silicone- or wax-treated paper. The main package body can also be made of more then one layer of material, such as an inner and an outer layer.

The package includes a main package body and a flap. The main package body completely encloses the articles contained therein. The main package body can have any suitable size and shape. One embodiment has a substantially rectangular parallelepipedal shape. The main package body is provided with a secondary opening member, which, when opened, provides an opening in the main package body through which the articles can be conveniently removed. The opening typically has a length to width ratio of from about 1 to about 12.

In one embodiment the opening in the main package body is essentially linear. “Essentially linear opening” herein means that the opening has a length larger than its width. In the package of the present invention the essentially linear opening has a length to width ratio in the range of from about 5 to about 9. The linearity of the opening provided by the secondary opening member can translate into a very low area of the actual opening compared to a circular or ellipsoidal opening. This may provide a smaller area for contaminants to enter the package after opening the secondary opening member. In certain embodiments, when closed, the secondary opening member seals the articles contained in the main package body in a dust proof manner. Suitable means for providing the secondary opening member include, but are not limited to tear tapes, zip-lock closure means, adhesive tapes, weakness or score lines in the material of the main package body or other known means for closing or sealing the particular packaging material chosen.

In another embodiment, the opening in the main package body is two-dimensional for allowing convenient dispensing of the articles contained in the package. The opening can have any size and shape, as long as it can be effectively fully covered by the primary opening member, i.e. the flap. Exemplary, non-limiting shapes are circular, ellipsoidal, rectangular, square or triangular.

The flap is connected to the main package body along a line of connection. “Connection” herein does not imply that the flap has to be a separate piece of material which needs to be joined to the main package body. Thus, “line of connection” is to be interpreted as the line along which the flap extends away from the main package body when the flap is in its open configuration. It is also within the scope of the present invention that main package body and flap are made of one integral piece of material. Further, the flap can have any suitable size and shape. In one configuration, the line of connection has a width about the same as the width of the main package body along the line of connection. The flap can be pivoted between an open and a closed configuration. In the closed configuration the free end of the flap can be secured to the main package body by an adhesive tape, a Velcro-type hook-loop fastener or the like. The line of connection between the main package body and the flap is arranged such that, when the flap is in its open position, it exposes the opening provided by the secondary opening member, and when the flap is in its closed position, it covers the opening provided by the secondary opening member in the main package body. This is especially advantageous when the secondary opening member is opened because the flap then covers the opening created thereby and thus aids the hygienic protection of the articles still contained in the main package body.

The flap can be joined or substantially non-joined to the secondary opening member. In the first case, opening of the flap and opening of the secondary opening member may be essentially independent from each other. In the latter case, opening of both primary (flap) and secondary opening member can be coupled.

The flap can be joined to the secondary opening member such that when the flap is opened, the secondary opening member also opens and exposes the opening in the main package body. This can be facilitated by bonds or otherwise connecting, in some fashion, the flap and the secondary opening member.

In other embodiments of the present invention the flap is arranged to not cover the secondary opening member prior to use. This for instance refers to the shelf life of the package. In such embodiments, the flap may not cover the secondary opening member, but instead be oriented in another direction, such as, towards the backside of the package. When the package is then used, i.e. opened, the flap is de-joined from the backside of the package and pivoted over the opening generated by opening the secondary opening member. The free end of the flap can optionally be secured in this position by the means disclosed herein for this purpose. Of course, when an adhesive is used therefore, a protective cover like a release liner may be desirable to preserve the adhesive in functional state over longer time intervals.

The means for securing the flap in its closed position can be colored for serving as identification means. This is particularly relevant in case the flap is transparent or translucent.

In an embodiment where the main package body is made of two layers of material the flap may be an integral part of the outer layer. For instance, the flap can be formed by lines of perforations in the outer layer of material, which are broken by the user when opening the outer layer. By the opening generated thereby, the inner layer is exposed in the region of the opening. The secondary opening member may be a perforation in the exposed area of the inner layer of material.

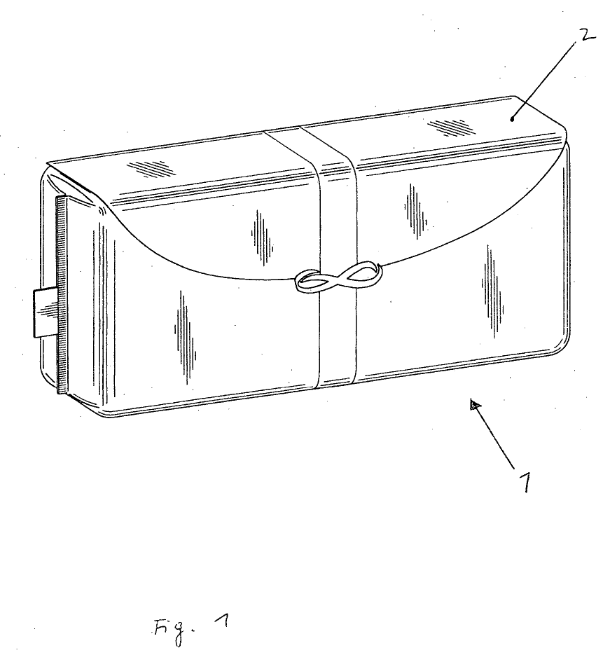

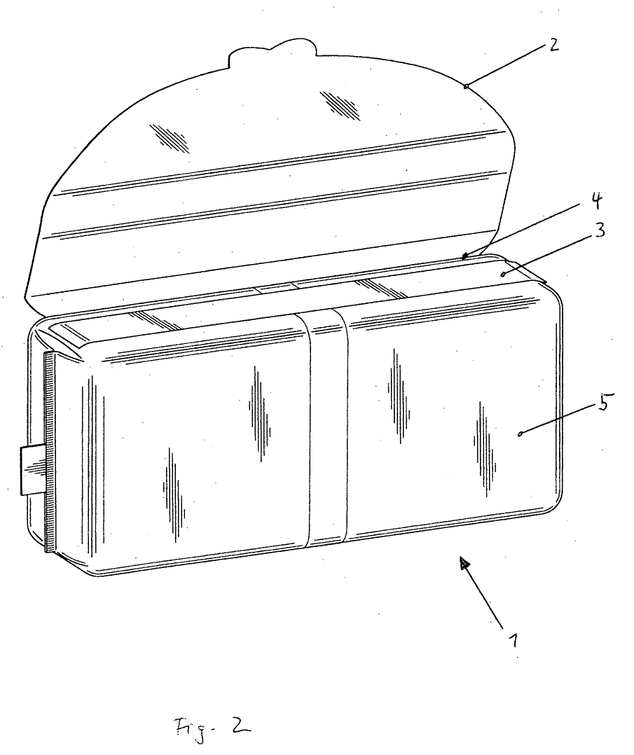

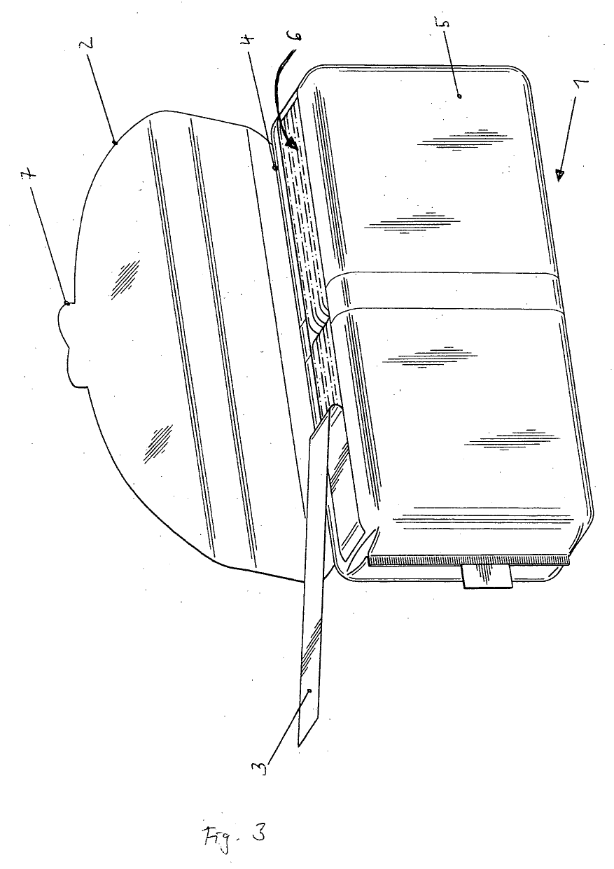

One embodiment of the present invention is illustrated in FIGS. 1-3. FIG. 1 illustrates the package in completely closed state, whereas FIG. 2 illustrates the package with the flap 2 opened to expose the secondary opening member 3. FIG. 3 then illustrates the package with the secondary opening member 3 partially opened. In the embodiment shown, the main package body 1 has a substantially parallelepipedal shape. Further, the line of connection 4 of the flap 2 to the main package body 1 extends parallel to a side edge of the main package body 5 and has the same length of the edge between the line of connection 4 and an adjacent side edge of the main package body 5. The secondary opening member 3 is shown to extend between the line of connection 4 and an adjacent side edge of the main package body 5. The secondary opening member 3 is shown executed as a tear tape having a free end for grasping. The tear tape is arranged close and generally parallel to the line of connection 4 such that the flap 2, when closed, effectively completely covers the tear tape. The flap 2 shown has a semi-circular shape with a piece of adhesive tape 7 connected to its free end for securing to the main package body 5. The absorbent articles contained in the package shown are arranged in two stacks.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A package for a multiplicity of absorbent articles arranged in at least one stack, the package comprising:
a main package body enclosing the at least one stack;
a reclosable flap hingedly connected to the main package body along a line of connection, wherein the flap can be pivoted between an open and a closed configuration; and
a secondary opening means being capable of providing an opening in the main package body, wherein the secondary opening means is arranged in relation to the line of connection such that the flap in the closed configuration covers the opening provided by the secondary opening means.

2. The package of claim 1, wherein the opening has a length to width ratio of from about 1 to about 12.

3. The package of claim 2, wherein the opening has a length to width ratio of from about 5 to about 9.

4. The package of claim 3, wherein the opening provided by the secondary opening means is essentially linear.

5. The package of claim 1, wherein the reclosable flap can be reclosed by an adhesive tape or a hook and loop mechanical fastener.

6. The package of claim 1, wherein the package is made from a flexible polymeric film material such as polyethylene, polypropylene, cellophane, polyester or polyvinyl chloride.

7. The package of claim 1, wherein the secondary opening means is provided by a tear tape or a zip-lock closure or a weakness or score line.

8. The package of claim 1, wherein the absorbent articles are selected from diapers, sanitary napkins, panty liners, tampons, incontinence pads, breast pads and perspiration pads.
